(19) United States
(12) Patent Application Publication
(10) Pub. No.: US 2009/0105775 A1
(43) Pub. Date: Apr. 23, 2009

(54) CANNULA WITH LATERAL ACCESS AND DIRECTIONAL EXIT PORT

(76) Inventors: David MITCHELL, Spartanburg, SC (US); William WALTERSDORFF, Hernando, MS (US)

Correspondence Address: KENYON & KENYON LLP, 1500 K STREET N.W., SUITE 700, WASHINGTON, DC, 20005, US

(21) Appl. No.: 12/198,711

(22) Filed: Aug. 26, 2008

Related U.S. Application Data
(63) Continuation-in-part of application No. 11/976,016, filed on Oct. 19, 2007.

(51) Int. Cl. A61B 17/58

(52) U.S. Cl. 606/86 R

(57) ABSTRACT

The present invention generally provides a cannula system that is readily maneuverable in an operating room setting, can be used to expose different instrumentalities to a target site, and has directional capabilities to allow the user to treat multiple quadrants or areas of a target site. The present invention provides cannula systems including these different instrumentalities as well as methods of operating these cannula systems and methods of treating orthopedic conditions using these cannula systems.

### CROSS REFERENCE TO RELATED APPLICATIONS

This application claims priority to and is a continuation-in-part application Ser. No. 11/976,016, filed on Oct. 19, 2007, the entirety of which is hereby incorporated by reference.

### TECHNICAL FIELD

The present invention generally relates to cannulas and cannula systems. More specifically, the present invention relates to cannulas and cannula systems having an inner lumen which laterally deflects an orthopedic device out of a distal end of the cannula.

### BACKGROUND

There are many different orthopedic conditions that require surgical intervention. For example, bone fractures are a very common orthopedic problem that can occur because of a number of factors, such as injury, disease or progressive age. One type of surgical procedure used to treat fractures of the spine is vertebroplasty. Vertebroplasty involves injecting liquid bone cement into the interstices of the weakened bone under pressure. The bone cement subsequently hardens to fix the vertebral body. Another process is kyphoplasty, in which a mechanical bone tamping device is used to elevate the vertebral body. An orthopedic cement is then injected into the space created by the bone tamp. Specifically, a bone tamping device, such as a balloon, can be placed into the intervertebral body and inflated so that a cavity is formed in the weakened bone. This cavity can then be filled with a more viscous form of bone cement.

Another type of orthopedic condition is degenerative disc disease, which can involve degeneration and age-related changes in the macroscopic, histologic and biochemical composition and structure of the annulus fibrosus and/or the nucleus pulposus of an intervertebral disc. There are numerous surgical treatment options for painful degenerative disc disease that have ranged in the past from interbody fusions to total disc replacement. Another, more recent option is plasma disc decompression which involves removing tissue from the nucleus pulposus using low temperature plasma excision.

Many of the orthopedic tools used in these procedures and other orthopedic procedures involve complex, high profile components. In addition, many are not completely controllable by the user. For example, in balloon kyphoplasty the balloons used to create the cavity can expand along the path of least resistance forming an unusual or asymmetrical cavity which inhibits or compromises the ideal placement of the cement. Therefore, the dimensions of a balloon created cavity are largely beyond the control of the user and more or less dependent upon the extent of disruption of the architecture of the pathologic bone. Furthermore, a problem associated with current orthopedic tool placement systems used in many of these procedures is that they do not accommodate the vertical height limitations present in the operating room during the procedure. For example, because of the fluoroscopic imaging devices that are above the orthopedic tool placement systems, a user has limited vertical space to maneuver instruments through the tool placement systems.

Therefore, a more controllable, lower profile orthopedic tool and accompanying placement system that is also designed to accommodate the user during performance of the surgical procedure is needed.

### SUMMARY

In an embodiment, the present invention provides a cannula system that allows for directional placement of an orthopedic tool as well as an entry port that can provide a user with more maneuverability in handling the orthopedic tool during a surgical procedure. A cannula of a cannula system of the present invention generally comprises a handle and a cannula shaft. The handle comprises a handle body having a proximal portion and a distal portion. The cannula shaft comprises an elongate tubular body having a proximal end depending from the distal portion of the handle body, a distal end terminating in a pointed tip, and a longitudinal axis extending therethrough. The handle further comprises a first entry port in fluid communication with a first lumen. The first entry port can be located on the side (as shown in FIG. 2) and/or the top (as shown in FIG. 2A) of the handle, for example. The cannula shaft further has an inner wall defining a channel that has a proximal end and a distal end. The proximal end of the channel is in fluid communication with the first lumen of the handle body and the distal end of the channel is in fluid communication with a side distal exit port. The distal end of the channel is also spaced apart from the distal end of the elongate body. The inner wall is configured to laterally deflect the channel at the channel's distal end with respect to the longitudinal axis of the elongate body to transition the channel's distal end to the side distal exit port. A cannula system of these embodiments further comprises an orthopedic surgical tool sized to be inserted into the first entry port of the handle and the channel of the cannula shaft. A cannula system of these embodiments further comprises one or more spacers, each spacer having a proximal contact surface, a distal contact surface, and a through hole extending through the proximal contact surface and the distal contact surface.

In an embodiment, the present invention provides a cannula system that allows for directional placement of an orthopedic tool as well as an entry port that can provide a user with more maneuverability in handling the orthopedic tool during a surgical procedure. A cannula of a cannula system of the present invention generally comprises a handle and a cannula shaft. The handle comprises a handle body having a proximal portion and a distal portion. The cannula shaft comprises an elongate tubular body having a proximal end depending from the distal portion of the handle body, a distal end terminating in a pointed tip, and a longitudinal axis extending therethrough. The handle further comprises a first entry port in fluid communication with a first lumen The cannula shaft further has an inner wall defining a channel that has a proximal end and a distal end. The proximal end of the channel is in fluid communication with the first lumen of the handle body and the distal end of the channel is in fluid communication with a side distal exit port. The distal end of the channel is also spaced apart from the distal end of the elongate body. The inner wall is configured to laterally deflect the channel at the channel's distal end with respect to the longitudinal axis of the elongate body to transition the channel's distal end to the side distal exit port. A cannula system of these embodiments further comprises an orthopedic surgical tool sized to be inserted into the first entry port of the handle and the channel of the cannula shaft.

In another embodiment, the present invention provides a cannula system including a cannula that comprises a handle and a cannula shaft. The handle comprises a handle body having a proximal portion and a distal portion. The handle further comprises a top entry port in fluid communication with a first lumen having a first longitudinal axis extending therethrough and a side entry port in fluid communication with a second lumen having a second longitudinal axis extending therethrough which intersects with the first longitudinal axis of the first lumen. The cannula shaft comprises an elongate body having a proximal end, a distal end, and a longitudinal axis extending therethrough. The proximal end of the elongate body extends from the distal portion of the handle body and the distal end of the elongate body terminates in a pointed tip. The channel shaft further has an inner wall defining a channel, the channel having a proximal end and a distal end. The proximal end of the channel is in fluid communication with the first and second lumens of the handle and the distal end of the channel is spaced apart from the distal end of the elongate body. The distal end of the channel is also in fluid communication with a side distal exit port. The inner wall is configured to laterally deflect the channel at its distal end with respect to the longitudinal axis of the elongate body of the cannula shaft to transition the channel's distal end to the side distal exit port. The cannula system further comprises a deflector that is selectably moveable into the first lumen of the handle through a lateral opening. The deflector has a surface that is angled or curved with respect to the first and second longitudinal axes to guide an orthopedic tool accessed through the side entry port down the second lumen of the handle. A cannula system in accordance with these embodiments further comprises an orthopedic surgical tool sized to be inserted into the top or side entry port of the handle and the channel of the cannula shaft. A cannula system of these embodiments further comprises one or more spacers, each spacer having a proximal contact surface, a distal contact surface, and a through hole extending through the proximal contact surface and the distal contact surface.

In another embodiment, the present invention provides a cannula system including a cannula that comprises a handle and a cannula shaft. The handle comprises a handle body having a proximal portion and a distal portion. The handle further comprises a top entry port in fluid communication with a first lumen having a first longitudinal axis extending therethrough and a side entry port in fluid communication with a second lumen having a second longitudinal axis extending therethrough which intersects with the first longitudinal axis of the first lumen. The cannula shaft comprises an elongate body having a proximal end, a distal end, and a longitudinal axis extending therethrough. The proximal end of the elongate body extends from the distal portion of the handle body and the distal end of the elongate body terminates in a pointed tip. The channel shaft further has an inner wall defining a channel, the channel having a proximal end and a distal end. The proximal end of the channel is in fluid communication with the first and second lumens of the handle and the distal end of the channel is spaced apart from the distal end of the elongate body. The distal end of the channel is also in fluid communication with a side distal exit port. The inner wall is configured to laterally deflect the channel at its distal end with respect to the longitudinal axis of the elongate body of the cannula shaft to transition the channel's distal end to the side distal exit port. The cannula system further comprises a deflector that is selectably moveable into the first lumen of the handle through a lateral opening. The deflector has a surface that is angled or curved with respect to the first and second longitudinal axes to guide an orthopedic tool accessed through the side entry port down the second lumen of the handle. A cannula system in accordance with these embodiments further comprises an orthopedic surgical tool sized to be inserted into the top or side entry port of the handle and the channel of the cannula shaft. In these embodiments, the orthopedic surgical tool is selected from the group consisting of a bone tamping device comprising a rod depending from a handle and a beveled tip located at the distal-most end of the rod or a biopsy tube comprising a tube body having at least a distal portion that is flexible enough to laterally deflect out of the side distal exit port of the cannula.

The invention may be embodied in numerous devices and through numerous methods and systems. The following detailed description, taken in conjunction with the annexed drawings, discloses examples of the invention. Other embodiments, which incorporate some, all or more of the features as taught herein, are also possible.

### BRIEF DESCRIPTION OF THE DRAWINGS

The present invention will become more fully understood from the detailed description given hereinbelow and the accompanying drawings which are given by way of illustration only, and thus are not limitative of the present invention, and wherein:

FIG. 1 is a side view of a cannula according to an embodiment of the present invention.

FIG. 2 is a side cross-sectional view of a cannula according to an embodiment of the present invention.

FIG. 2A is a side cross-sectional view of a cannula shaft according to an embodiment of the present invention having a single, top entry port.

FIG. 3 is a side cross-sectional view of a cannula shaft according to an embodiment of the present invention.

FIG. 4 is a side cross-sectional view of a cannula shaft according to an embodiment of the present invention.

FIG. 5 is a side cross-sectional view of a cannula shaft according to an embodiment of the present invention.

FIG. 6 is a side view of a cannula according to an embodiment of the present invention.

FIG. 7 is a side cross-sectional view of a cannula shaft according to an embodiment of the present invention.

FIG. 8 is a side cross-sectional view of a cannula shaft according to an embodiment of the present invention.

FIG. 9 is a side cross-sectional view of a cannula shaft according to an embodiment of the present invention.

FIG. 10 is a side cross-sectional view of a cannula shaft according to an embodiment of the present invention.

FIG. 11 is a top view of a handle of a cannula according to an embodiment of the present invention.

FIG. 12 is a side cross-sectional view of a cannula shaft according to an embodiment of the present invention with a deflector inserted into a lateral opening of the handle.

FIG. 13 is a side cross-sectional view of a cannula shaft according to an embodiment of the present invention.

FIG. 14 is a side view of a bone tamp device according to an embodiment of the present invention.

FIG. 14A is an isometric view of a bone tamp device (with a beveled distal tip) and a cannula according to an embodiment of the present invention.

FIG. 14B is an expanded view around circle 14B of the cannula and bone tamp device of FIG. 14A.

FIG. 14C is a side view of the portion of the cannula and bone tamp device in circle 14B of FIG. 14A.

FIG. 15 is a side view of a stylet according to an embodiment of the present invention.

FIG. 15A is an isometric view of a cannula carrying a biopsy tube with a plurality of apertures according to an embodiment of the present invention.

FIG. 15B is an expanded view around circle 15B of the cannula and biopsy tube of FIG. 15A.

FIG. 15C is an isometric cross-sectional view of the cannula and biopsy tube of FIG. 15B.

FIG. 15D is an isometric view of a biopsy tube having longitudinal slots according to an embodiment of the present invention.

FIG. 16 is a side view of a catheter according to an embodiment of the present invention.

FIG. 16A is an isometric view of a cannula having entry ports with luer lock connectors in accordance with an embodiment of the present invention.

FIG. 16B is an isometric cross-sectional view of the handle of the cannula of FIG. 16A.

FIG. 16C is an isometric view of a cannula, orthopedic device, and spacer in accordance with an embodiment of the present invention.

FIG. 16D is an isometric view of the cannula, orthopedic device, and spacer of FIG. 16B, showing the spacer limiting the insertion depth of the orthopedic device.

FIG. 16E is an isometric view of a cannula system having multiple spacers in accordance with an embodiment of the present invention.

FIG. 17 is a schematic illustration of a fractured vertebra.

FIG. 18 is a schematic illustration of a cannula carrying a bone tamp according to an embodiment of the present invention inserted into the fractured vertebra of FIG. 17.

FIG. 19 is a schematic illustration of the bone tamp device of FIG. 18 that has exited a side distal exit port of the cannula to tamp one side of the vertebra.

FIG. 20 is a schematic illustration of the cannula of FIG. 18 rotated 180°.

FIG. 21 is a schematic illustration of the cannula of FIG. 20 where the bone tamp has exited the side distal exit port of the cannula to tamp the opposing side of the vertebra.

### DETAILED DESCRIPTION

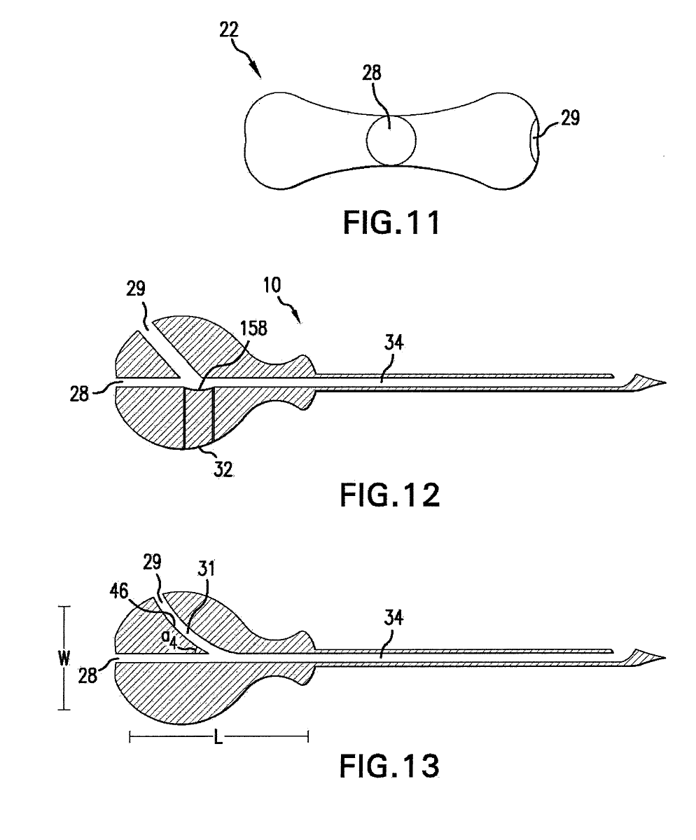

As used herein, the terms “side,” “top” and “down” are described with respect to the cannula system as seen from a top plan view (such as shown in FIG. 11).

In general, the present invention provides a cannula system that is readily maneuverable in an operating room setting, can be used to expose different instrumentalities to a target site, and has directional capabilities to allow the user to treat multiple quadrants or areas of a target site. Since the cannula system has particular application in the orthopedic setting, the target site is often bone.

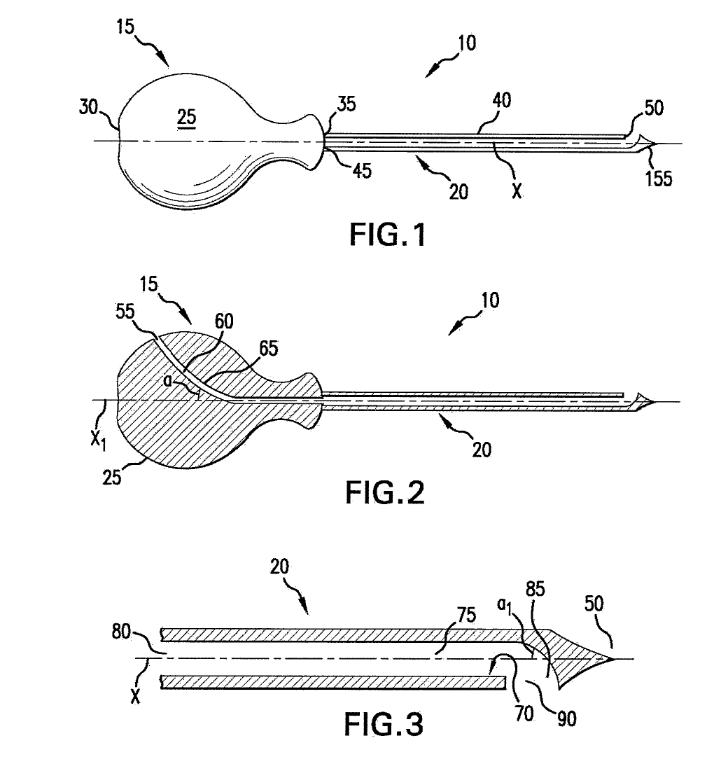

Specifically, referring to FIG. 1, in an embodiment, the present invention provides a cannula system that includes a cannula 10 comprising a handle 15 and a cannula shaft 20. Handle 15 comprises a handle body 25 having a proximal portion 30 and a distal portion 35. Cannula shaft 20 comprises an elongate tubular body 40 having a proximal end 45 and a distal end 50. Proximal end 45 depends from distal portion 35 of handle body 25 and distal end 50 terminates in a tip 155. In preferred embodiments, tip 155 is a closed tip. Tip 155 can have any suitable configuration to cut into tissue such as, for example, a diamond shape, as shown in FIG. 3 or a beveled or threaded tip, the latter of which may allow for slower insertion of the cannula into bone. Elongate tubular body 40 further has a longitudinal axis X extending through proximal end 45 and distal end 50.

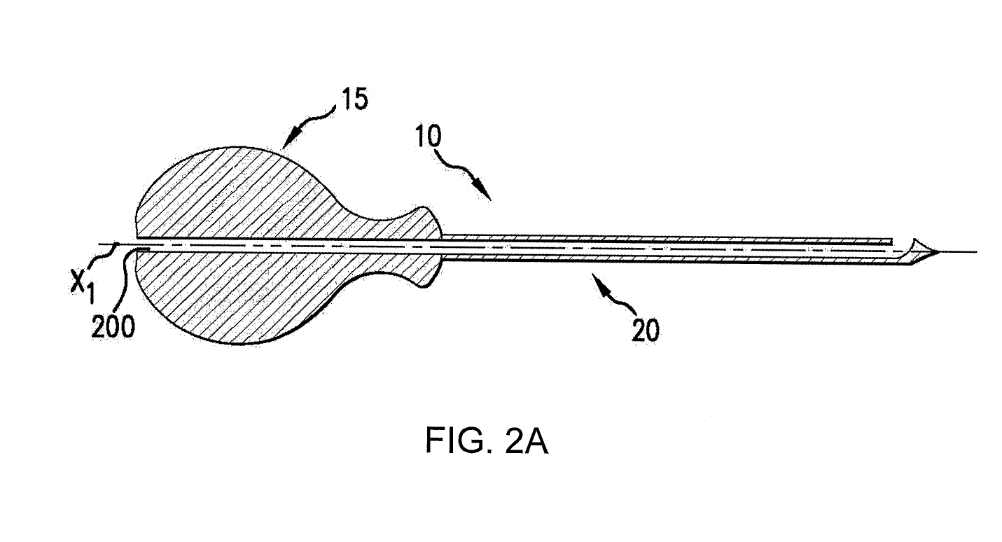

Referring to FIG. 2, in an embodiment, handle 15 further comprises a first entry port 55 in fluid communication with a first lumen 60. In certain embodiments, such as that shown in FIG. 2, first lumen 60 has a partial section 65 that is curved or angled with respect to an imaginary center line X₁, such center line extending through handle body 25 and being aligned with the longitudinal axis X of cannula shaft 20. In these embodiments, the partial section 65 of first lumen 60 forms an acute angle a with respect to imaginary center line X₁ of handle body 25. This configuration of handle 25 allows a user to laterally insert any suitable instrumentality into first lumen 60 via first entry port 55, which, in turn, allows the user to maneuver the device without facing any vertical height constraints that exist in prior art cannula systems where the users could only access the cannula shaft via a top entry port in the handle. The entry port need not be located on a side of the cannula handle (as shown in FIG. 2) and may be located on the top portion of the handle (as shown by top entry port 201 in FIG. 2A). Additionally, in certain embodiments the handle may include two or more entry ports comprising any combination of side and/or top entry ports. For example, the handle may comprise two side entry ports (as shown in FIG. 7) or a side entry port and a top entry port (as shown in FIG. 13). The entry port(s) could also be located on different locations on the handle.

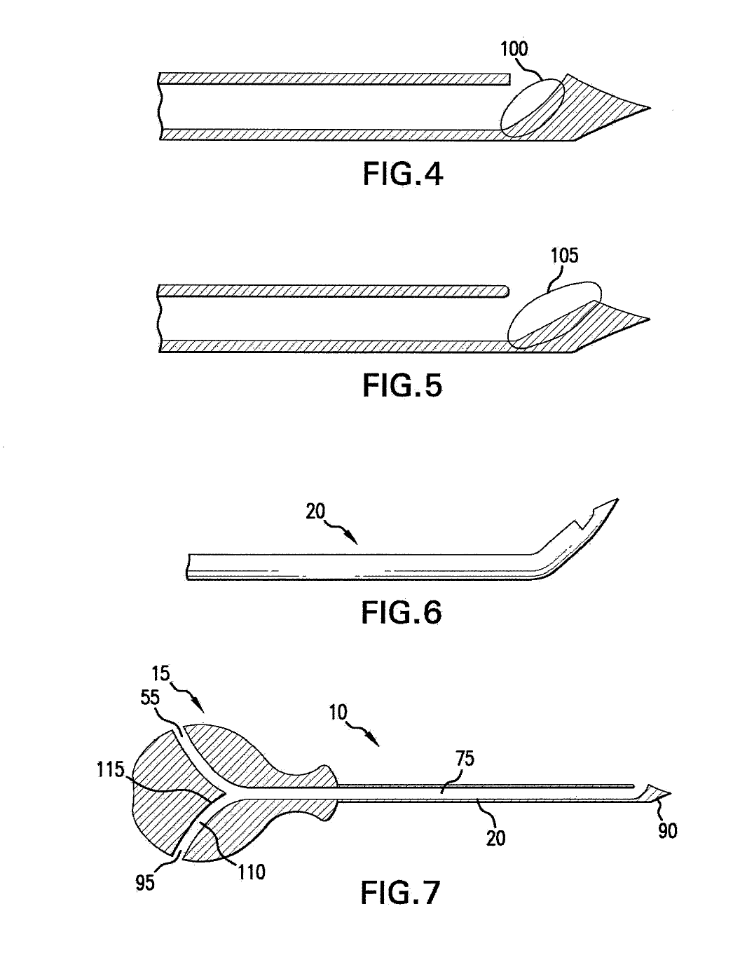

Referring to FIG. 3, cannula shaft 20 of cannula 10 has an inner wall 70 defining a channel 75 that is in fluid communication with first lumen 60 of handle 15 (such first lumen and channel collectively referred to herein with respect to this embodiment as a bore). Channel 75 has a proximal end 80 and a distal end 85. Proximal end 80 is adjacent to and in fluid communication with first lumen 60 of handle body 25 and distal end 85 is adjacent to and in fluid communication with a first side distal exit port 90. Distal end 85 of channel 75 is also spaced apart from distal end 50 of elongate body 40 of cannula shaft 20. As seen in FIG. 3, inner wall 70 is configured to laterally defect channel 75 at the channel's distal end 85 with respect to longitudinal axis X of elongate body 40. Therefore, inner wall 70 has a curvature 100, as more clearly seen in FIG. 4 or an angled portion 105, as more clearly seen in FIG. 5, to transition channel 75 to first side distal exit port 90. Referring back to FIG. 3, preferably distal end 85 of channel 75 forms an acute angle a₁ with respect to longitudinal axis X of elongate body 40.

This side distal exit port of cannula shaft 20 allows a user to insert an instrument through the bore to access one side of a target site of the body, such as a fractured vertebra. Upon performance of a designated procedure with the instrument, the user simply needs to remove the instrument, rotate the cannula a desired degree to access another side of the target site of the body, re-insert the instrument through the bore, and perform the designated procedure with the instrument on another side of the target site. The side distal exit port provides a user with directionality during the procedure so that the user can access different areas of the target site.

Referring to FIG. 6, in certain embodiments, cannula shaft 20 is pre-bent near its distal end to provide an additional degree of directionality.

Referring to FIG. 7, in another embodiment, the present invention provides a cannula system including a cannula 10 where handle 15 further comprises a second entry port 95. In certain embodiments (such as the embodiment shown in FIG. 7), entry ports 55 and 95 are located on opposite sides of handle body 15. Second entry port 95 is in fluid communication with a second lumen 110 that, in certain embodiments, may have a partial section 115 that is curved or angled with respect to an imaginary center line X₁, such center line extending through handle body 25 and being aligned with the longitudinal axis X of cannula shaft 20. Furthermore, second lumen 110 is in fluid communication with channel 75 of cannula shaft 20 (such second lumen and channel referred to herein with respect to this embodiment as a bore). Channel 75, in turn, is in fluid communication with side distal exit port 90. Such a design allows a user to insert an instrument through either first or second entry port depending, for example, on which side is more accessible or comfortable for the user. Alternatively, such a design allows a user to insert one type of instrument through the first entry port and another type of instrument through the second entry port.

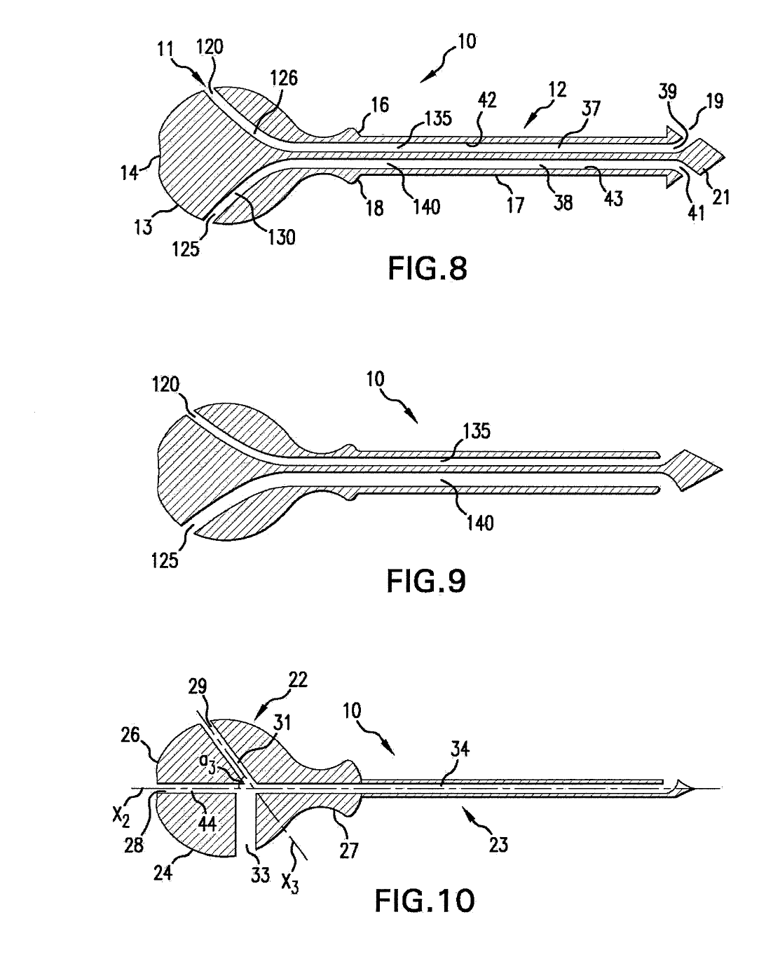

Referring to FIG. 8, in another embodiment, the present invention provides a cannula system including a cannula with dual lumens. Specifically, in this embodiment, cannula 10 comprises a handle 11 and a cannula shaft 12. Handle 11 comprises a handle body 13 having a proximal portion 14 and a distal portion 16. Cannula shaft 12 comprises an elongate tubular body 17 having a proximal end 18 and a distal end 19. Proximal end 18 depends from distal portion 16 of handle body 13 and a distal end 19 terminates in a pointed tip 21. Handle 11 comprises a first side entry port 120 on one side of handle body 13 and a second side entry port 125 on an opposite side of handle body 13. As with the embodiment described with respect to FIG. 7, first side entry port 120 is in fluid communication with a first lumen 126 that has at least a partial section that is curved or angled and second side port 125 is in fluid communication with a second lumen 130 that has at least a partial section that is curved or angled. However, unlike FIG. 7, first and second lumens 126 and 130 are in fluid communication with separate, parallel first and second channels 135 and 140, respectively, of cannula shaft 12 (such first lumen 126 and first channel 135 collectively referred to herein with respect to this embodiment as a first bore and such second lumen 130 and second channel 140 collectively referred to as a second bore). In the embodiment illustrated in FIG. 8, the first and second bores 37 and 38 have the same general diameter. However, in other embodiments, it may be preferable for one of the bores to be larger than the other bore as illustrated in FIG. 9. Although both entry ports are shown in FIGS. 7-9 as side entry ports, this is not the case for all embodiments, and one or both of the first and second entry ports may be located on the top of the handle of the cannula (similar to entry port 200 in FIG. 2A or the configuration shown in FIG. 13).

In certain embodiments, such as that shown in FIG. 8, first channel 135 of cannula shaft 12 is defined by a first inner wall 42 and is in fluid communication with and adjacent to first lumen 126 of handle body 13 at one end, as stated above, and in fluid communication with and adjacent to a first side distal exit port 39 at another end. Similarly, second channel 140 is defined by a second inner wall 43 and is in fluid communication with second lumen 130 at one end, as stated above, and in fluid communication with a second side distal exit port 41 at another end. As seen in FIG. 8, first and second inner walls 42 and 43 are configured to laterally defect respective channels 135 and 140 at the respective channel's distal end with respect to longitudinal axis X of elongate body 17 to transition the respective channels to the respective side distal exit ports.

The opposing side distal exit ports of cannula shaft 12 in this embodiment allows a user to insert an instrument through the first bore of cannula 10 to access one side of a target site of the body, such as a fractured vertebra. Upon performance of a designated procedure with the instrument, the user need not rotate the cannula to access the opposing side of the target site. Rather, the user simply needs to insert the same instrument or an identical instrument through the second bore to access the opposing side of the target site. Alternatively or in addition, the user can use the first and second entry ports 125 and 120 to insert different types of instrumentalities.

Referring to FIG. 10, in another embodiment, the present invention provides a cannula system including cannula 10 comprising a handle 22 and a cannula shaft 23. Handle 22 comprises a handle body 24 having a proximal portion 26 and a distal portion 27. The handle further comprises a top entry port 28 in fluid communication with a first lumen 44 having a first longitudinal axis X₂ extending therethrough. Handle 22 further comprises a side entry port 29 in fluid communication with a second lumen 31 having a second longitudinal axis X₃ extending therethrough which intersects with the first longitudinal axis X₂ of first lumen 44. FIG. 11 provides a top plan view of handle 22 to illustrate the location of top entry port 28 and side entry port 29 in this embodiment. Preferably, second longitudinal axis X₃ intersects with first longitudinal axis X₂ at an angle, a₃ of 90° or less.

Referring to FIG. 12, the cannula system further comprise a deflector 32 that is selectively moveable into first lumen 44 through a lateral opening 33 (shown also in FIG. 10). Deflector 32 has a surface 158 that is angled or curved with respect to first and second longitudinal axes X₂ and X₃. Referring back to FIG. 10, cannula shaft 23 is configured as described with respect to FIG. 2 such that a channel 34 of cannula shaft 23 is in fluid communication with first and second lumens 44 and 31 of handle 22 (such channel and first lumen collectively referred to herein as a first bore and such channel and second lumen collectively referred to as a second bore for purposes of this embodiment). In such an embodiment, the user has a choice whether to use the top entry port, as is done conventionally, to insert an instrument through the cannula or to use the side entry port, as described above. If the user decides to use side entry port 29, then the user can insert deflector 32 into lateral opening 33 so that the angled or curved surface 158 of the deflector can guide the instrument down channel 34.

Referring to FIG. 13, in another embodiment, cannula 10 can be designed to avoid the need for a deflector. For example, second lumen 31 can be defined by an inner wall 46 that has a curvature sufficient to inherently guide an instrument down lumen 31 to channel 34. Specifically, an instrument will follow the arc of inner wall 46 to channel 34. Preferably, the angle a4 is 45° or less.

Regarding exemplary measurements of a cannula according to embodiments of the present invention, in certain embodiments, the handle has a length L of between about 4 to 5 inches, preferably about 2 to 3 inches. In certain embodiments, the handle has a width W, as measured at its maximum width, of between about 0.25 inches to 0.50 inches. In certain embodiments, cannula shaft has length between about 4 and 8 inches, preferably about 6 inches and a diameter of about 11 to 17 gauge, and preferably about 13 gauge.

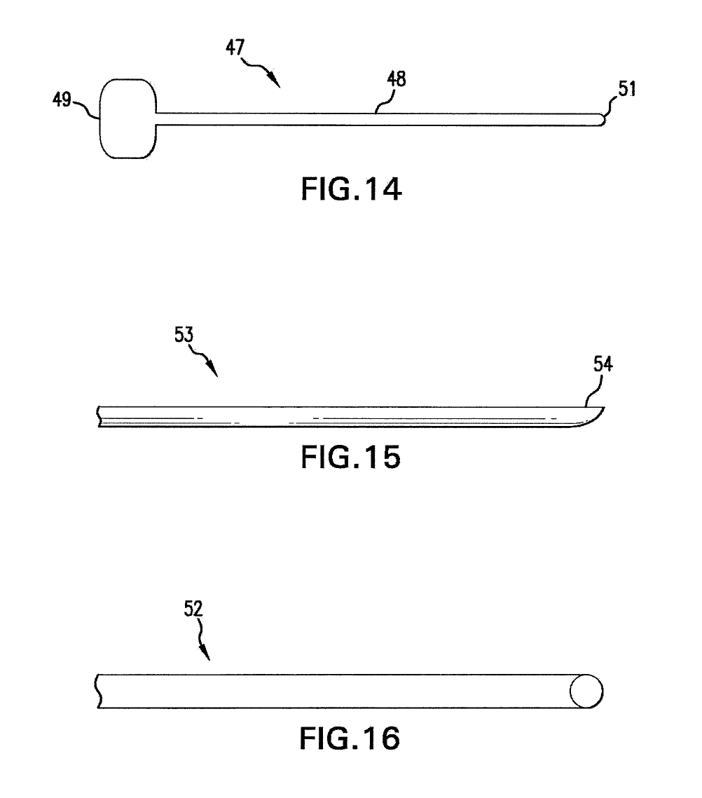

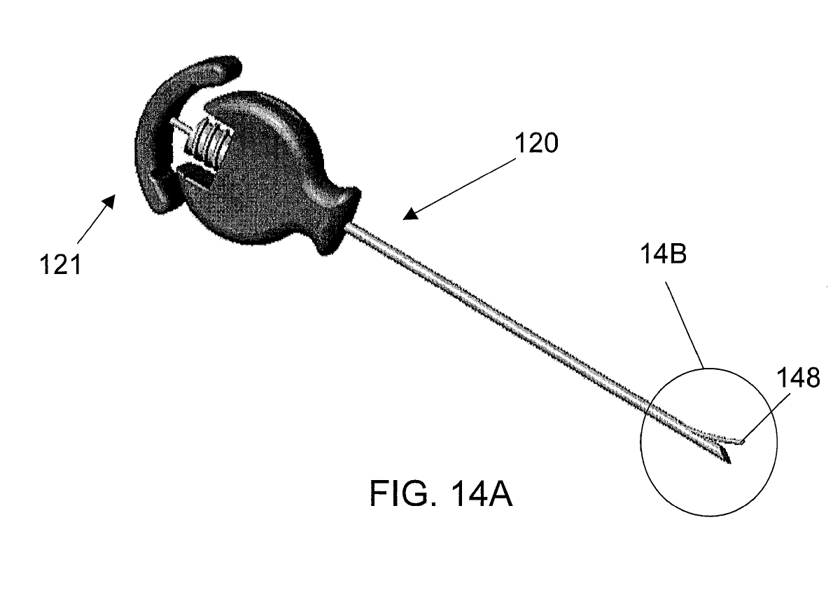

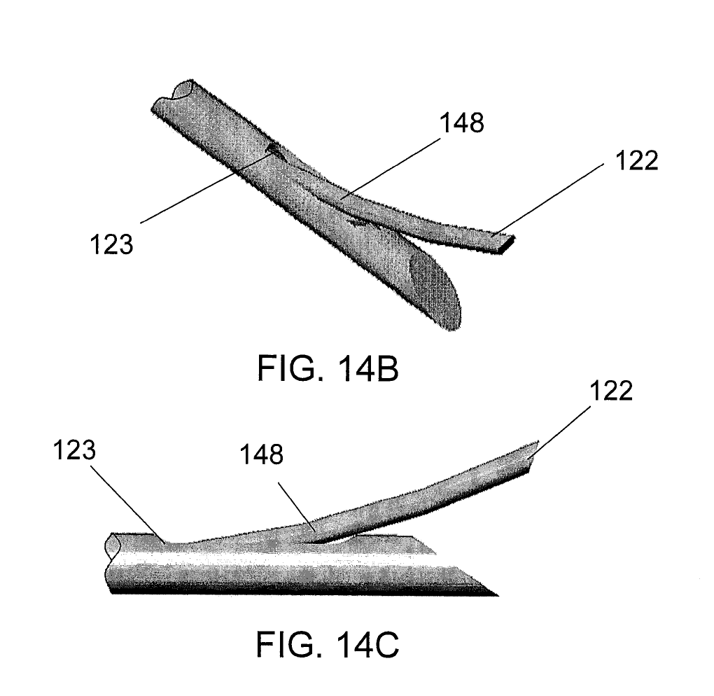

The above described cannula be used with a variety of different instruments to perform various functionalities. For example, a cannula can be used with a bone tamp to provide a mechanical means by which to lift or elevate bone to reduce a bone fracture, for example. An exemplary illustration of a bone tamp device 47 is provided in FIG. 14. This bone tamp device comprises a rod 48 depending from a handle 49. Rod 48 has a length longer than that of any of the bores of a cannula so that, in use, rod 48 can extend past the side distal exit port of the cannula to access the target site. Preferably, handle 49 has a flattened configuration for ease of manipulation during use. In embodiments where bone tamp device 47 is inserted in a side curved or angled lumen (as described above), rod 48 is fabricated from a flexible material to allow rod 48 to bend as it is urged down the curved or angled lumen. Non-limiting examples of suitable flexible materials include a flexible metal or elastomeric polymer. Non-limiting examples of suitable materials include titanium, expandable polytetrafluorethylene (ePTFE), or polyetheretherketone (PEEK).

The distal end tip 51 of rod 48 is shown in FIG. 14 as being rounded. However, the tip could be flat, sharp, threaded, beveled, or have other configurations so long as the bone tamp can be used to elevate bone. For example, the distal end tip of rod 148 of bone tamp device 121 may have the beveled configuration shown in FIGS. 14A-14C to allow for better guidance of tamp device 121 out from the angled or curved distal exit port 123 of cannula 120 and to therefore increase the surface area of the bone being tamped. For example, as bone tamp device 121 is inserted into cannula 120, a beveled tip 122 of bone tamp device 121 allows for improved guidance through exit port 123 of cannula 120, as shown in FIGS. 14A and 14B. This beveled tip also allows the bone that is to be tamped to be exposed to a sufficient surface area of rod 148 as best seen in FIG. 14B (or to an increased surface area of rod 148 compared to a rod that does not have a beveled tip).

The other configurations of distal end tip 51 of bone tamp device 47 of FIG. 14 could also be tailored to match other functionalities for which the bone tamp may be used. For example, bone tamp device 47 could be used as an osteotome, for example, to cut a sclerotic lesion that otherwise prevents the bone tamp from elevating the rest of the bone. In such an embodiment, it may be desirable for the distal end of the bone tamp device to be sharp although this is not a necessity. To use the bone tamp device as an osteotome, the bone tamp can be inserted into a bore of a cannula and, upon reaching the site that is to be cut, urged out of a side distal exit port of the cannula at a distance, for example, of three to four millimeters. In order to cut the desired tissue, the handle of the cannula and the handle of the bone tamp device can be turned in concert causing the cannula and bone tamp to rotate, thereby allowing the bone tamp to cut the desired tissue.

In alternative embodiments, the bone tamp is not used as the osteotome, but rather a separate osteotome is used.

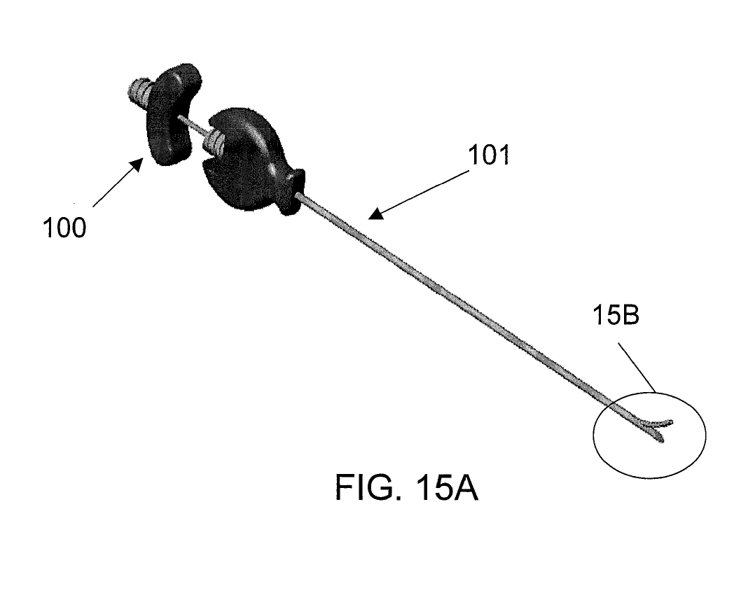

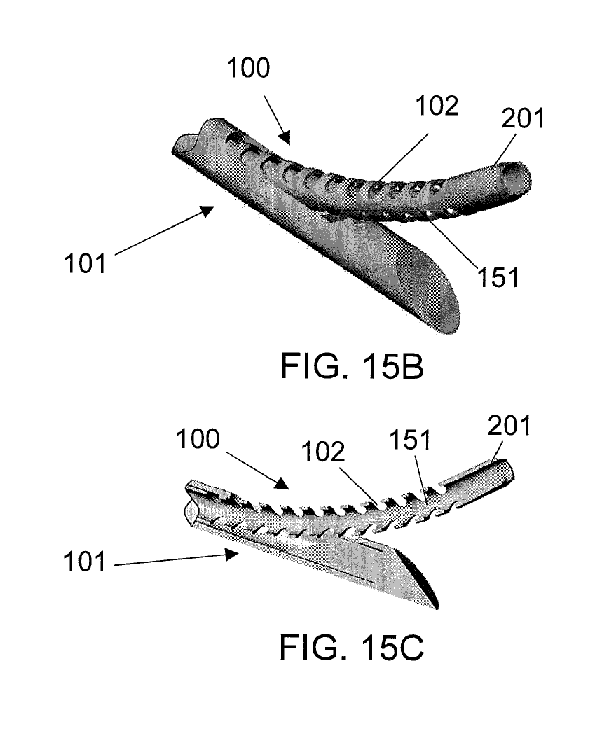

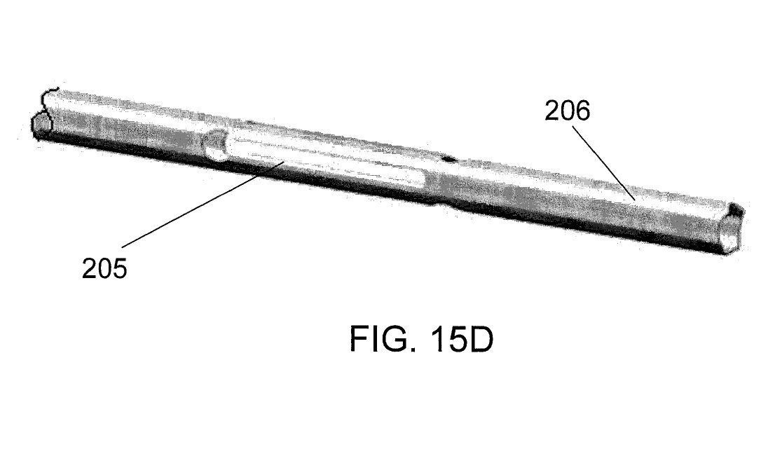

In addition or alternatively, the cannula can be used with or as a biopsy needle to aspirate fluid from the bone tissue and/or to retrieve bone marrow tissue itself. For this use, a stylet 53 with a sharp tip, as shown in FIG. 15, can be inserted into any of the above described bores of a cannula and urged through a side distal exit port of the shaft. Upon entering the bone marrow cavity, the stylet can be withdrawn and, using a syringe at the proximal end of the cannula (through either a top or side port), marrow can be aspirated under negative pressure. If it is desired to also retrieve a solid bone marrow specimen, then a cylindrical tube can be inserted into another bore of the cannula. In such a case, it is desirable for the another bore in which the cylindrical tube is inserted to be larger than the bore used to aspirate fluid from the bone marrow so that the larger bore can accommodate a larger diameter cylindrical tube. Such a configuration of a cannula where one bore is larger than another bore is shown in FIG. 9. Once the larger diameter cylindrical tube is positioned in the cannula, a stylet can be used to penetrate the bone cortex. The stylet can then be withdrawn and the larger diameter tube remaining in the another bore can be pushed further into the marrow causing a core of marrow to enter the tube. The tube can then withdrawn from the cannula and the core of marrow pushed out with a blunt probe through the tube lumen.

In the embodiment shown in FIG. 15, the distal end 54 of stylet 53 is shaped to match the curvature of the distal end of a cannula shaft of a cannula to fill the side distal exit port. Such a configuration may be useful to prevent debris from entering the cannula and also to increase the strength of the cannula so that the cannula will not bend when pressure is applied thereto (such as in the case of hammering the cannula) in certain circumstances. Of course, other shapes for the distal end of the stylet could also be used.

In certain other embodiments, such as those shown in FIGS. 15A-15C, a cannula 101 may be used with a biopsy tube 100 that includes a tube body 151 having at least a distal portion 201 that is sufficiently flexible to allow the biopsy tube to laterally deflect from the side exit port of the cannula. In certain embodiments, this flexibility is achieved via a plurality of recesses 102 defined by tube body 151. The recesses allow for additional flexibility in the biopsy tube, which may be fabricated from any suitable material. As shown in the accompanying figures, the recesses are located around the circumference of the tube body (as opposed to at the distal tip) and can extend along at least a portion of the length of the biopsy tube but at least toward the distal portion of the biopsy tube. The recesses may comprise a variety of configurations, including (but not limited to) rectangular shapes, circular shapes, or the ovular through holes shown in FIGS. 15A-15C. Although the recesses are shown as through holes in FIGS. 15A-15C, this is not true for all embodiments, and the recesses may simply be recessed portions of the tube body. As mentioned above, the recesses need not be located along the entire length of the tube body and may be located along only a portion of the length. Similarly, the recesses need not be located around the entire circumference of the tube body but may be located on only a top or bottom portion, for example.

In certain other embodiments, the biopsy tube may further comprise a plurality of longitudinal slots 205 defined by tube body 206 as shown in FIG. 15D. The slots may be of variable length and may be located at a distal portion of the tube body or elsewhere along the tube body. One of ordinary skill in the art will appreciate that the slots may comprise a broad range of configurations and locations, and any configuration of the slots that allows for some flexibility in the biopsy tube is possible.

In certain other embodiments, the biopsy tube may comprise means for flexing the biopsy tube (“flex means”). The flex means allow for the biopsy tube to be sufficiently flexible at at least its distal end such that the biopsy tube may laterally deflect from the side exit port of the cannula. Non-limiting examples for the flex means include the plurality of recesses and plurality of longitudinal slots described above.

In certain embodiments, at least a portion of the biopsy tube is comprised of a flexible material (for example, at the distal end). Non-limiting examples of suitable flexible materials include amorphous thermoplastic polyetherimides (such as Ultem™), shape memory materials (such as Nitinol), nylon, or medical grade plastic. In addition, materials with a phase transition temperature approximately equal to the temperature of the human body (for example, a material that becomes soft or pliable at approximately 97.6-99.6° F.) may be used. Other flexible materials known in the art and suitable for use with a biopsy tube and that allow the biopsy tube to laterally deflect from the exit port of the cannula may also be used.

In addition or alternatively, a cannula of the present invention can be used to deliver a bone material to a bone fracture site in order to augment the bone. The bone material can be a bone graft material, a bone paste and/or a bone morphogenetic protein (BMP). Bone graft materials are well known in the art and include both natural and synthetic materials. For example, the bone graft material can be an autologous or autograft, allograft, xenograft, or synthetic bone graft. The bone graft can be in the form of corticocancellous bone chips. BMPs are also well known in the art and include BMP-2, BMP-3, BMP-4, BMP-5, BMP-6 (VGR-1), BMP-7 (OP-1), BMP-8, BMP-9, BMP-10, BMP-11, BMP-12, BMP-13, BMP-14, BMP-15. Preferred BMPs are any of BMP-2, BMP-3, BMP-4, BMP-5, BMP-6, and BMP-7. The bone paste can be a cement or ceramic material including, for example, polymethylmethacrylate. The bone material can be introduced through any of the above described entry ports of the cannulas of the present invention via mechanisms known in the art, such as syringes and filler tubes that are attachable or otherwise able to be received by the entry ports.

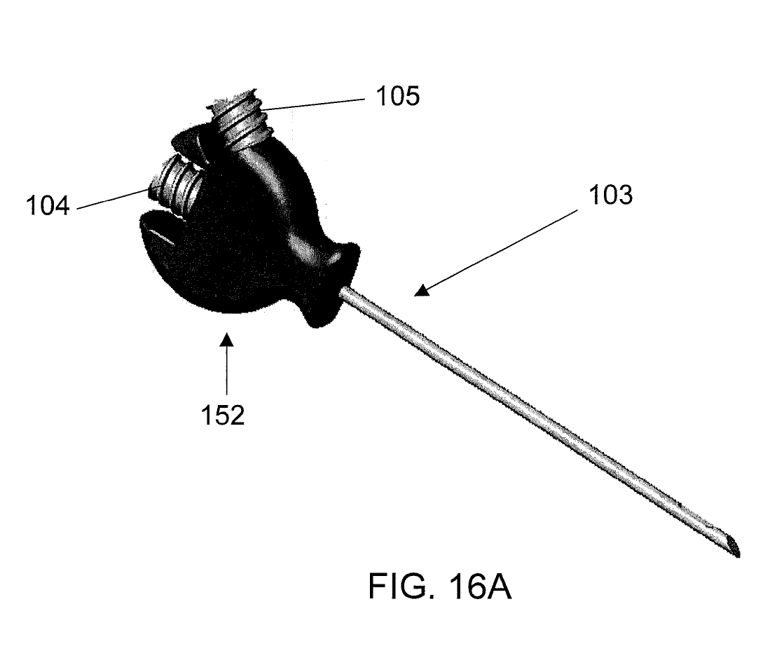

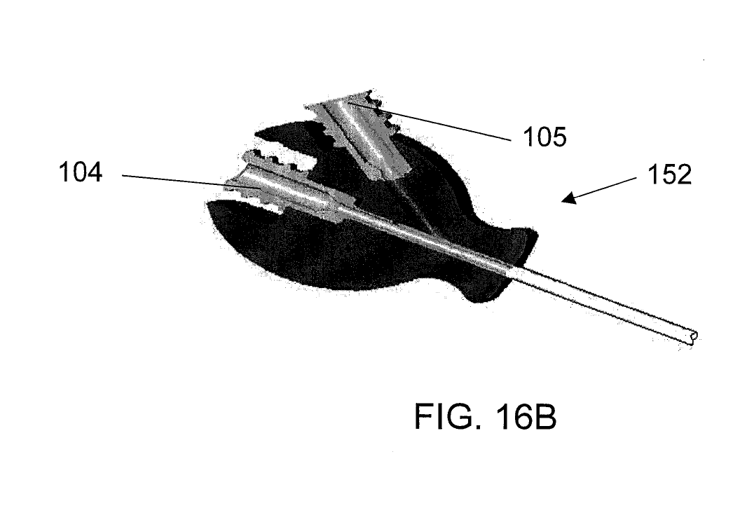

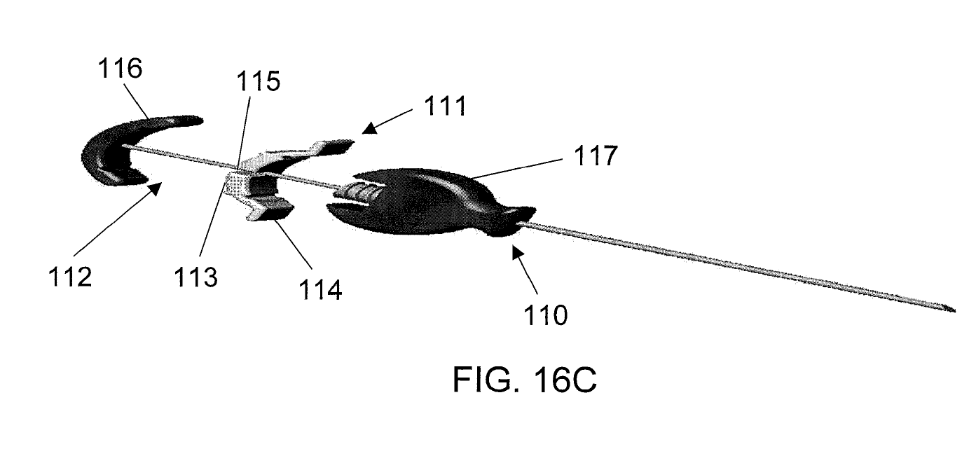

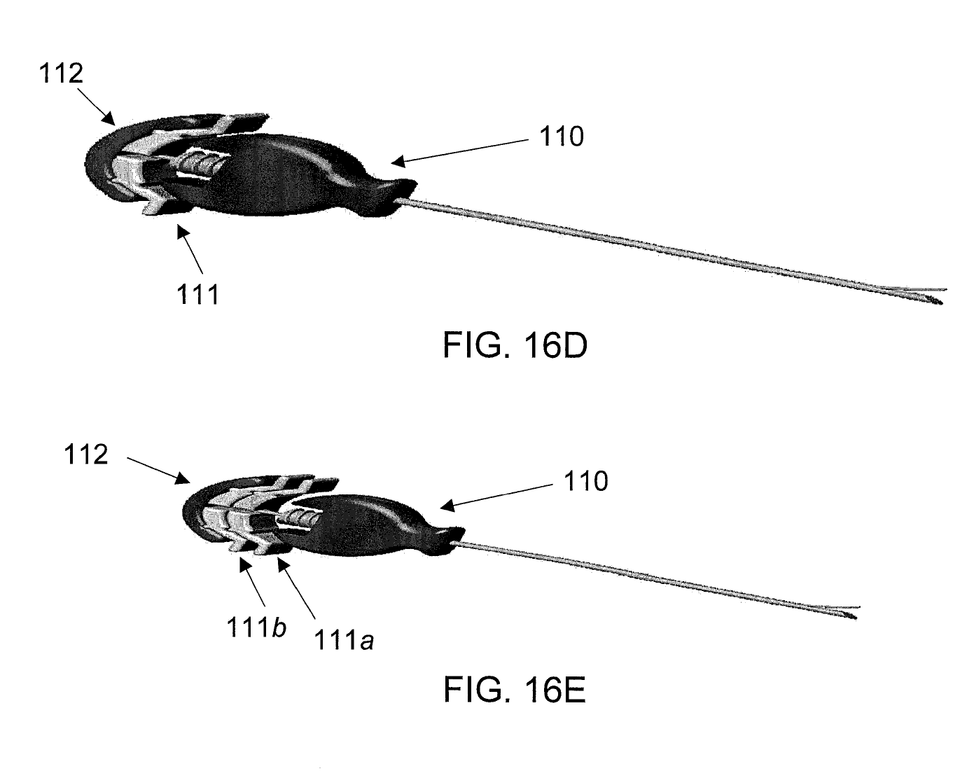

In addition or alternatively, a cannula of the present invention can be used with a catheter 52 as shown in FIG. 16 to deliver a therapeutic agent to a target site. Non-limiting examples of therapeutic agents include anti-microbial agents, antibiotics or stem cells. Such therapeutic agents can be delivered separately to the target site or can be incorporated into a bone material (described above) and delivered to the target site.

In addition or alternatively, a cannula of the present invention can be used to deliver a viscoelastic polymer to a disc to replace other components of the disc, such as the nucleus pulposis.

A cannula of the present invention can be used with other type of orthopedic tools used in spinal surgery such as devices that deliver thermal or heat energy including radiofrequency waves and/or laser beams. The cannulas could also be used to delivery non-thermal energy such as low energy radiofrequency waves for plasma disc decompression. Specifically, the cannulas of the present invention can be used to deliver radio wave signals through an electrode introduced into a bore of the cannula to the nucleus pulposus. The radio waves produce a low-temperature ionized gas or plasma that breaks up molecular bonds in the nucleus, removing tissue volume, which results in disc decompression.

A cannula of the present invention may also further comprise luer lock connectors on one or more of the cannula's entry ports. The addition of leur lock connectors allows for increased flexibility and ease in attaching additional instrumentation to the cannula system. In certain embodiments, such as the system shown in FIGS. 16A and 16B, a cannula 103 has a handle 152 and luer lock connectors 104 and 105 on top and side entry ports. The luer lock connectors can be either male or female, as needed, and may be configured to receive various other instrumentation. The luer lock connectors on a particular cannula need not all have the same configuration, and both male and female luer lock connectors may be used on the same cannula.

A cannula system of the present invention may further comprise one or more spacers used to control the insertion depth of an orthopedic device (such as, for example, a bone tamping device). Turning to FIG. 16C, an exemplary cannula system in accordance with these embodiments is shown. Cannula 110 is shown with spacer 111 and orthopedic device 112. Spacer 111 comprises proximal contact surface 113, distal contact surface 114, and through hole 115. The proximal contact surface may be configured to rest on or within at least a portion of a handle of the orthopedic device, such as, for example, the configuration of proximal contact surface 113 that allows surface 113 to fit within orthopedic device handle 116 in FIG. 16B. Similarly, the distal contact surface may be configured to rest on or within at least a portion of a handle of the cannula, such as, for example, the configuration of distal contact surface 114 that allows surface 114 to rest on a portion of cannula handle 117. The through hole of the spacer extends through the proximal contact surface and the distal contact surface, allowing a rod or tube of the orthopedic device to pass through the spacer. The through hole may comprise a variety of configurations including, but not limited to, circular, ovular, or rectangular holes or the groove (115) shown in FIG. 16C.

The spacers may be configured to limit the insertion depth of the orthopedic device as needed. Accordingly, the spacers may comprise a variety of widths and shapes. As shown in FIG. 16D, in an applied position, a spacer is used to prevent the orthopedic device from extending distally beyond a certain point.

Although only one spacer is shown in FIGS. 16C and 16D, this is not true for all embodiments, and in certain other embodiments two or more spacers may be used, such as in the cannula system of FIG. 16E. In these embodiments, the proximal or distal contact surfaces of a spacer may be configured to rest on or within at least a portion of the proximal or distal contact surfaces of an adjacent spacer (similar to spacers 111a and 111b shown in FIG. 16E). In this way, the spacers may “stack” on one another. Each spacer need not have the same configuration, and spacers within the same cannula system may comprise different widths, shapes, or through hole and contact surface configurations.

Any of the above-described processes and tools can be used with any of the other above-described processes and tools in cannula systems of the present invention. Further, other orthopedic tools used in orthopedic surgeries could be used in addition to or as an alternate to the above-described orthopedic tools.

The systems and methods of the present invention can be used in a variety of orthopedic procedures to treat a variety of orthopedic conditions. For example, the systems of the present invention can be used procedures such as disc decompression, discectomy, stabilization (fusion), kyphoplasty and vertebroplasty. In a preferred embodiment, the systems of the present invention are used to treat fractures. The fractures can be in various parts of the body, such as fractures of the shoulder, arms, wrists, hands and fingers; fractures of the spine; fractures of the hips and pelvis; and fractures of the legs, knees and feet. In a particularly preferred embodiment, the systems and methods of the present invention are used to treat vertebral fractures. Such vertebral fractures (as well as other spine conditions that can be addressed by a cannula system of the present invention) can be caused by a variety of etiologies such as, for example, scoliosis, herniated disc, spondylolisthesis, sciatica, spondylitis, spondylosis, spinal stenosis, trauma, tumor reconstruction or degenerative disc diseases. Of course the above listed conditions and etiologies are only exemplary and the systems of the present invention are not necessarily limited to any particular use.

Preferably, the systems of the present invention access the fractures via a minimally invasive route, such as percutaneously. In embodiments where the fracture that is treated is a spinal fracture, the systems can access the spine through various approaches such as a posterior approach or an anterior approach.

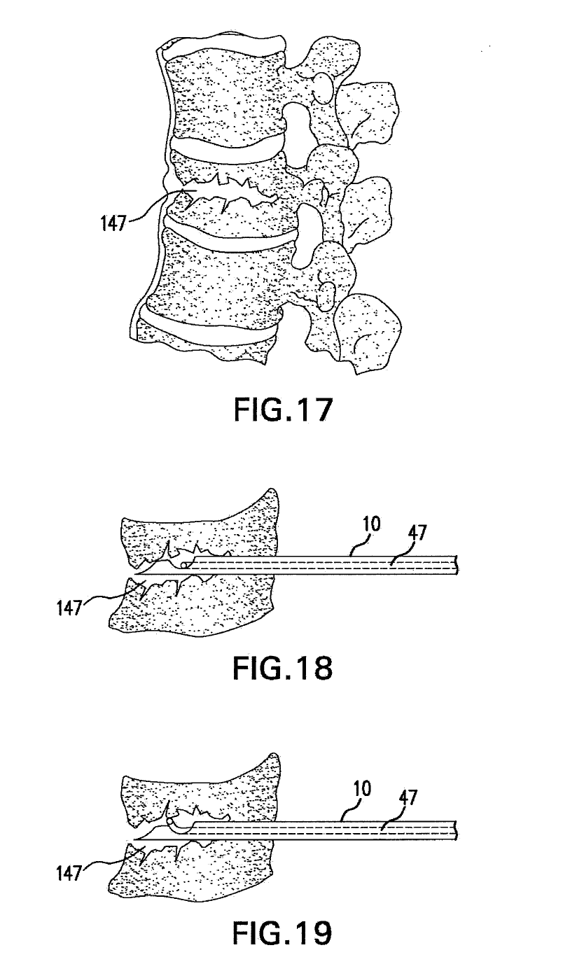

An exemplary surgical procedure will now be described using a cannula system of the present invention for vertebral body fracture reduction on a patient with a collapsed vertebral body, as shown in FIG. 17, who is in need of reduction of the fracture 147 followed by filling in of the void created by such reduction. Under general anesthesia, the patient is positioned prone on a radiolucent operating table and biplanar fluoroscopy is used to visualize the fractured vertebral body. The procedure could also be done under sedation or using locally applied numbing medicine. A stylet is inserted into a cannula 10 of the present invention and the cannula is inserted into the body in a percutaneous fashion to the level of the pedicle or any other desired position on the vertebral body. This process is followed fluoroscopically to ensure proper positioning of the cannula. Once the cannula is inserted to the desired location in the vertebral body, the stylet can be removed and a biopsy can be obtained by removing the cannula stylet and inserting a plastic or metal cylindrical tube with an auger type end into the bone to retrieve a desired sample. This same procedure can be repeated on the contralateral side of the vertebral body if desired or needed.

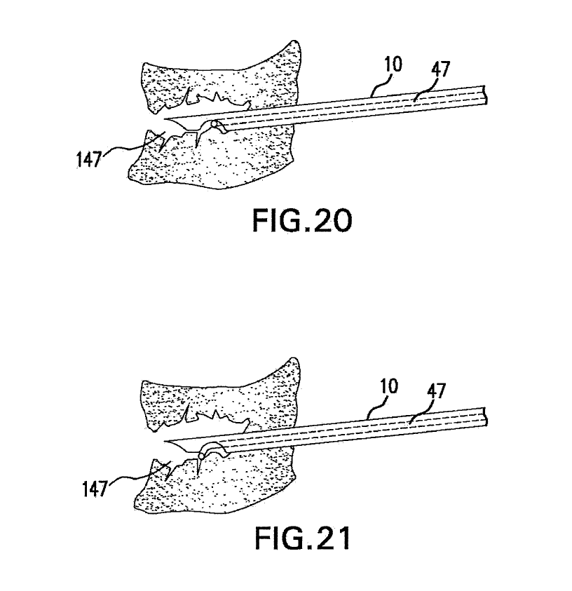

To perform reduction of the vertebral body, a bone tamp device 47 is inserted into the cannula as shown in FIG. 18. As shown in FIG. 19, tamp device 47 is deflected by the angled or curved side distal exit port of the cannula and becomes directional by means of turning the cannula handle, which can have directional markings on the handle. By withdrawing and inserting the inner device multiple times, the tamp can be used to reduce the compressed vertebral bone and this reduction can be observed by means of fluoroscopy. The directional capability of the cannula will allow for reduction of multiple quadrants or areas of the vertebral body. For example, as shown in FIGS. 20 and 21, the cannula can be rotated to tamp the opposing side of fracture 147.

The reduction procedure can create small voids that can be stabilized with cement or other materials capable of hardening or at least forming a stable construct onto which the fracture reduction can rest. In such an instance, a high viscosity bone cement is inserted into the vertebral body via the cannula. The bone tamp device and stylet are removed from the cannulas and the bone cement attachments are attached to the entry ports. This will allows directional placement of cement into the vertebral bodies at a slow rate with cement that is highly viscous thus allowing for visualization under fluoroscopy (as the cement would be radio opaque). After the cement is injected, the cannula is rotated to break any remaining cement ties with the cannula and then the cannula is withdrawn.

The foregoing description and examples have been set forth merely to illustrate the invention and are not intended as being limiting. Each of the disclosed aspects and embodiments of the present invention may be considered individually or in combination with other aspects, embodiments, and variations of the invention. Further, while certain features of embodiments of the present invention may be shown in only certain figures, such features can be incorporated into other embodiments shown in other figures while remaining within the scope of the present invention. In addition, unless otherwise specified, none of the steps of the methods of the present invention are confined to any particular order of performance. Modifications of the disclosed embodiments incorporating the spirit and substance of the invention may occur to persons skilled in the art and such modifications are within the scope of the present invention. Furthermore, all references cited herein are incorporated by reference in their entirety.

What is claimed is:

1. A cannula system comprising:
a cannula, comprising:
a handle and a cannula shaft, the handle comprising a handle body having a proximal portion and a distal portion, the cannula shaft comprising an elongate tubular body having a proximal end depending from the distal portion of the handle body, a distal end terminating in a pointed tip, and a longitudinal axis extending therethrough;
the handle further comprising a first entry port in fluid communication with a first lumen;
the cannula shaft further having an inner wall defining a first channel, the first channel having a proximal end and a distal end, the proximal end of the first channel in fluid communication with the first lumen of the handle body, the distal end of the first channel in fluid communication with a first side distal exit port and spaced apart from the distal end of the elongate body, the inner wall configured to laterally deflect the first channel at the first channel's distal end with respect to the longitudinal axis of the elongate body to transition the first channel's distal end to the first side distal exit port;
an orthopedic surgical tool sized to be inserted into the first entry port of the handle and the channel of the cannula shaft; and
one or more spacers, each spacer having a proximal contact surface, a distal contact surface, and a through hole extending through the proximal contact surface and the distal contact surface.

2. The cannula system of claim 1, wherein the one or more spacers comprise a plurality of spacers.

3. The cannula system of claim 1, wherein the orthopedic surgical tool is selected from the group consisting of a bone tamp device, a biopsy tube, a stylet, an electrode, a drug delivery catheter, an osteotome, or any combination thereof.

4. The cannula system of claim 1, wherein the handle of the cannula further comprises a second entry port in fluid communication with a second lumen.

5. The cannula system of claim 1, wherein the first entry port is located on a side of the handle of the cannula.

6. The cannula system of claim 1, wherein the first entry port is located on a top portion of the handle of the cannula.

7. A cannula system comprising:
a cannula, comprising:
a handle and a cannula shaft, the handle comprising a handle body having a proximal portion and a distal portion, the cannula shaft comprising an elongate tubular body having a proximal end depending from the distal portion of the handle body, a distal end terminating in a pointed tip, and a longitudinal axis extending therethrough;
the handle further comprising a first entry port in fluid communication with a first lumen;
the cannula shaft further having an inner wall defining a first channel, the first channel having a proximal end and a distal end, the proximal end of the first channel in fluid communication with the first lumen of the handle body, the distal end of the first channel in fluid communication with a first side distal exit port and spaced apart from the distal end of the elongate body, the inner wall configured to laterally deflect the first channel at the first channel's distal end with respect to the longitudinal axis of the elongate body to transition the first channel's distal end to the first side distal exit port;
an orthopedic surgical tool sized to be inserted into the first entry port of the handle and the channel of the cannula shaft.

8. The cannula system of claim 7, wherein the orthopedic surgical tool is a bone tamping device comprising a rod depending from a handle and having a proximal end and a distal end, the distal end terminating in a beveled tip.

9. The cannula system of claim 7, wherein the orthopedic surgical tool is a biopsy tube comprising a tube body and having at least a distal portion that is sufficiently flexible to allow the biopsy tube to laterally deflect from the first side distal exit port.

10. The cannula system of claim 9, wherein the biopsy tube further comprises means for flexing the biopsy tube.

11. The cannula system of claim 10, wherein the means for flexing the biopsy tube is a plurality of recesses defined by the tube body.

12. The cannula system of claim 10, wherein the means for flexing the biopsy tube is a plurality of longitudinal slots defined by the tube body.

13. The cannula system of claim 9, wherein the biopsy tube further comprises a plurality of recesses defined by the tube body.

14. The cannula system of claim 9, wherein the biopsy tube further comprises a plurality of longitudinal slots defined by the tube body.

15. The cannula system of claim 13, wherein the plurality of recesses defined by the tube body of the biopsy tube is a plurality of through holes.

16. The cannula of claim 9, wherein the at least a distal portion of the biopsy tube comprises a flexible material selected from the group consisting of: an amorphous thermoplastic polyetherimide a shape memory material, nylon, and a medical grade plastic.

17. The cannula system of claim 7, wherein the cannula further comprises a second entry port in fluid communication with a second lumen.

18. The cannula system of claim 7, wherein the first entry port is located on a side of the handle of the cannula.

19. The cannula system of claim 7, wherein the first entry port is located on a top portion of the handle of the cannula.

20. A cannula system, comprising:
a cannula comprising:
a handle and a cannula shaft, the handle comprising a handle body having a proximal portion and a distal portion and further comprising:
a top entry port in fluid communication with a first lumen having a first longitudinal axis extending therethrough;
a side entry port in fluid communication with a second lumen having a second longitudinal axis extending therethrough which intersects with the first longitudinal axis of the first lumen; and
the cannula shaft comprising an elongate body having a proximal end, a distal end, and a longitudinal axis extending therethrough, the proximal end of the elongate body depending from the distal portion of the handle body and the distal end of the elongate body terminating in a pointed tip, the elongate body further having:
an inner wall defining a channel, the channel having a proximal end and a distal end, the proximal end in fluid communication with the first and second lumens of the handle and the distal end of the channel in fluid communication with a side distal exit port, the distal end of the channel spaced apart from the distal end of the elongate body, the inner wall configured to lateral deflect the channel at its distal end with respect to the longitudinal axis of the elongate body of the cannula shaft to transition the channel's distal end to the side distal exit port;
a deflector that is selectably moveable into the first lumen through a lateral opening, the deflector having a surface that is angled or curved with respect to the first and second longitudinal axes;
an orthopedic surgical tool sized to be inserted into the top entry port and the side entry port of the handle and the channel of the cannula shaft;
one or more spacers, each spacer having a proximal contact surface, a distal contact surface, and a through hole extending through the proximal contact surface and the distal contact surface.

21. The cannula system of claim 20, wherein the orthopedic surgical tool is selected from the group consisting of a bone tamp device, a biopsy tube, a stylet, an electrode, a drug delivery catheter, an osteotome, or any combination thereof.

22. The cannula system of claim 20, wherein the one or more spacers comprise a plurality of spacers.

23. A cannula system, comprising:
a cannula comprising:
a handle and a cannula shaft, the handle comprising a handle body having a proximal portion and a distal portion and further comprising:
a top entry port in fluid communication with a first lumen having a first longitudinal axis extending therethrough;
a side entry port in fluid communication with a second lumen having a second longitudinal axis extending therethrough which intersects with the first longitudinal axis of the first lumen; and
the cannula shaft comprising an elongate body having a proximal end, a distal end, and a longitudinal axis extending therethrough, the proximal end of the elongate body depending from the distal portion of the handle body and the distal end of the elongate body terminating in a pointed tip, the elongate body further having:
an inner wall defining a channel, the channel having a proximal end and a distal end, the proximal end in fluid communication with the first and second lumens of the handle and the distal end of the channel in fluid communication with a side distal exit port, the distal end of the channel spaced apart from the distal end of the elongate body, the inner wall configured to lateral deflect the channel at its distal end with respect to the longitudinal axis of the elongate body of the cannula shaft to transition the channel's distal end to the side distal exit port;
a deflector that is selectably moveable into the first lumen through a lateral opening, the deflector having a surface that is angled or curved with respect to the first and second longitudinal axes;
an orthopedic surgical tool sized to be inserted into the top entry port and the side entry port of the handle and the channel of the cannula shaft;
wherein the orthopedic surgical tool is selected from the group consisting of:
a bone tamping device comprising a rod depending from a handle and having a proximal end and a distal end, the distal end terminating in a beveled tip; or
a biopsy tube comprising a tube body and a plurality of recesses defined by the tube body.

24. The cannula system of claim 23, wherein the orthopedic surgical tool is a biopsy tube and the plurality of recesses defined by the tube body of the biopsy tube is a plurality of through holes.
